(19) United States
(12) Patent Application Publication
(10) Pub. No.: US 2005/0084867 A1
(43) Pub. Date: Apr. 21, 2005

(54) Hybridization and scanning apparatus

(76) Inventors: Michael P. Caren, Palo Alto, CA (US); Richard J. Pittaro, San Carlos, CA (US); Carol T. Schembri, San Mateo, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/687,397

(22) Filed: Oct. 15, 2003

(51) Int. Cl. C12Q 1/68; G06K 9/00; C12M 1/34

(52) U.S. Cl. 435/6; 435/287.2; 382/128

(57) ABSTRACT

Apparatus and methods are disclosed relating to linear arrays. An embodiment of an apparatus comprises a rotatable support for one or more linear arrays, a mechanism for rotating the support, and a device for examining the linear arrays for the results of chemical reactions. Each of the linear arrays comprises a plurality of features for conducting the chemical reactions. Optionally, the apparatus may comprise a heater for heating the linear arrays. Optionally, the apparatus may comprise a fluid dispensing device for dispensing processing fluids such as, for example, wash solutions.

### BACKGROUND OF THE INVENTION

The present invention relates to apparatus and methods for conducting chemical and biological analyses using linear arrays. More particularly, the invention relates to apparatus and methods for hybridization reactions using linear microarrays of biopolymers in which molecular probes on a surface of a solid substrate selectively bind target molecules provided in a solution. The invention has utility in fields relating to biology, chemistry and biochemistry. The invention has particular application to the area of analyzing the results of hybridization reactions involving nucleic acids and proteins.

Determining the nucleotide sequences and expression levels of nucleic acids (DNA and RNA) is critical to understanding the function and control of genes and their relationship, for example, to disease discovery and disease management. Analysis of genetic information plays a crucial role in biological experimentation. This has become especially true with regard to studies directed at understanding the fundamental genetic and environmental factors associated with disease and the effects of potential therapeutic agents on the cell. Such a determination permits the early detection of infectious organisms such as bacteria, viruses, etc.; genetic diseases such as sickle cell anemia; and various cancers. New methods of diagnosis of diseases, such as AIDS, cancer, sickle cell anemia, cystic fibrosis, diabetes, muscular dystrophy, and the like, rely on the detection of mutations present in certain nucleotide sequences. This paradigm shift has lead to an increasing need within the life science industries for more sensitive, more accurate and higher-throughput technologies for performing analysis on genetic material obtained from a variety of biological sources.

Unique or misexpressed nucleotide sequences in a polynucleotide can be detected by hybridization with a nucleotide multimer, or oligonucleotide, probe. Hybridization reactions between surface-bound probes and target molecules in solution may be used to detect the presence of particular biopolymers. Hybridization is based on complementary base pairing. When complementary single stranded nucleic acids are incubated together, the complementary base sequences pair to form double stranded hybrid molecules. These techniques rely upon the inherent ability of nucleic acids to form duplexes via hydrogen bonding according to Watson-Crick base-pairing rules. The ability of single stranded deoxyribonucleic acid (ssDNA) or ribonucleic acid (RNA) to form a hydrogen bonded structure with a complementary nucleic acid sequence has been employed as an analytical tool in molecular biology research. An oligonucleotide probe employed in the detection is selected with a nucleotide sequence complementary, usually exactly complementary, to the nucleotide sequence in the target nucleic acid. Following hybridization of the probe with the target nucleic acid, any oligonucleotide probe/nucleic acid hybrids that have formed are typically separated from unhybridized probe. The amount of oligonucleotide probe in either of the two separated media is then tested to provide a qualitative or quantitative measurement of the amount of target nucleic acid originally present.

Such reactions form the basis for many of the methods and devices used in the field of genomics to probe nucleic acid sequences for novel genes, gene fragments, gene variants and mutations. The ability to clone and synthesize nucleotide sequences has led to the development of a number of techniques for disease diagnosis and genetic analysis. Genetic analysis, including correlation of genotypes and phenotypes, contributes to the information necessary for elucidating metabolic pathways, for understanding biological functions, and for revealing changes in genes that confer disease. Many of these techniques generally involve hybridization between a target nucleotide sequence and a complementary probe, offering a convenient and reliable means for the isolation, identification, and analysis of nucleotides. The surface-bound probes may be oligonucleotides, peptides, polypeptides, proteins, antibodies or other molecules capable of reacting with target molecules in solution.

Direct detection of labeled target nucleic acid hybridized to surface-bound polynucleotide probes is particularly advantageous if the surface contains a mosaic of different probes that are individually localized to discrete, known areas of the surface. Such ordered arrays of probes are commonly referred to as ‘biochip” arrays. Biochip arrays containing a large number of oligonucleotide probes have been developed as tools for high throughput analyses of genotype and gene expression. Oligonucleotides synthesized on a solid support recognize uniquely complementary nucleic acids by hybridization, and arrays can be designed to define specific target sequences, analyze gene expression patterns or identify specific allelic variations.

In one approach, cell matter is lysed, to release its DNA as fragments, which are then separated out by electrophoresis or other means, and then tagged with a fluorescent or other label. The resulting DNA mix is exposed to an array of oligonucleotide probes, whereupon selective attachment to matching probe sites takes place. The array is then washed and imaged so as to reveal for analysis and interpretation the sites where attachment occurred.

One typical method involves hybridization with probe nucleotide sequences immobilized in an array on a substrate having a surface area of typically less than a few square centimeters. The substrate may be glass, fused silica, silicon, plastic or other material; in particular, it may be a glass slide, which has been treated to facilitate attachment of the probes. The mobile phase, containing reactants that react with the attached probes, is placed in contact with the substrate, covered with another slide, and placed in an environmentally controlled chamber such as an incubator. Normally, the reactant targets in the mobile phase diffuse through the liquid to the interface where the complementary probes are immobilized, and a reaction, such as a hybridization reaction, then occurs. The mobile phase targets may be labeled with a detectable tag, such as a fluorescent tag, or chemiluminescent tag, or radioactive label, so that the reaction can be detected. The location of the signal in the array provides the target identification. The hybridization reaction typically takes place over a time period of seconds up to many hours.

Biochip arrays have become an increasingly important tool in the biotechnology industry and related fields. These binding agent arrays, in which a plurality of binding agents are synthesized on or deposited onto a substrate in the form of an array or pattern, find use in a variety of applications, including gene expression analysis, drug screening, nucleic acid sequencing, mutation analysis, and the like. Substrate-bound biopolymer arrays, particularly oligonucleotide, DNA and RNA arrays, may be used in screening studies for determination of binding affinity and in diagnostic applications, e.g., to detect the presence of a nucleic acid containing a specific, known oligonucleotide sequence.

The pattern of binding by target molecules to biopolymer probe spots on the biochip forms a pattern on the surface of the biochip and provides desired information about the sample. Hybridization patterns on biochip arrays are typically read by optical means, although other methods may also be used. For example, laser light in the Agilent Technologies Inc. GeneArray Scanner excites fluorescent molecules incorporated into the nucleic acid probes on a biochip, generating a signal only in those spots on the biochip that have a target molecule bound to a probe molecule, thus generating an optical hybridization pattern. This pattern may be digitally scanned for computer analysis. Such patterns can be used to generate data for biological assays such as the identification of drug targets, single-nucleotide polymorphism mapping, monitoring samples from patients to track their response to treatment, and assess the efficacy of new treatments.

One type of linear array is a one-dimensional array of features bound in a non-diffusive manner to a surface usually located on the inside of an enclosed microchannel. The order of the features identifies each feature, which allows selective identification of target molecules. One such linear array is disclosed in U.S. Pat. No. 5,804,384 (Muller, et al.). The devices of Muller, et al., consist of a tube containing a linear array of specific binding elements that each have capture probes specific for a target analyte.

There remains a need in the art for a simple, low cost apparatus and method for conducting chemical or biochemical reactions on a solid substrate.

### SUMMARY OF THE INVENTION

One embodiment of the present invention is an apparatus comprising a rotatable support for one or more linear arrays, a mechanism for rotating the support, and a device for examining the linear arrays for the results of chemical reactions. Each of the linear arrays comprises a plurality of features for conducting the chemical reactions. Optionally, the apparatus may comprise a heater for heating the linear arrays. Optionally, the apparatus may comprise a fluid dispensing device.

Another embodiment of the present invention is an apparatus for conducting and analyzing the results of hybridization reactions. The apparatus comprises a circular tray having a surface for supporting one or more channels, each of which may be within a housing, a mechanism for rotating the circular tray, and a scanning device for analyzing the results of the hybridization reactions. Each of the channels comprises one or more linear arrays. Each of the linear arrays comprises a plurality of biopolymers for conducting hybridization reactions. Optionally, the apparatus may comprise a heater for heating the linear arrays. Optionally, the apparatus may comprise a fluid dispensing device. Optionally, the apparatus may comprise a mechanism for examining the channel(s) for the presence of bubbles therein.

Another embodiment of the present invention is a method for conducting chemical reactions. A sample is introduced into a channel, which is some embodiments may be part of a housing, by capillary action and chemical reactions are conducted within the channel. The sample is incubated in the channel under conditions for carrying out the chemical reactions. The channel is then rotated to remove the sample from the housing.

Another embodiment of the present invention is a method for conducting hybridization reactions. In the method a sample is placed at an orifice of a channel, which may be part of a housing, comprising a linear array of features for hybridizing to analytes in the sample. The housing has internal dimensions to draw the sample therein by capillary action. The sample is incubated in the housing under conditions for carrying out the hybridization reactions. Then, the housing is rotated to remove the sample from contact with the features of the linear arrays by centrifugal force. Optionally, the method comprises examining the housing for the presence of bubbles therein. A plurality of the housings may be employed and may be disposed on a rotatable support. Optionally, the housing is pre-heated to a temperature for incubating the sample. The aforementioned approach may be employed to wash the interior of the housing.

### BRIEF DESCRIPTION OF THE DRAWINGS

The following figures are included to better illustrate the embodiments of the devices and techniques of the present invention. The figures are not to scale and some parts of the figures may be exaggerated for the purpose of illustrating certain aspects or embodiments of the present invention.

FIG. 1 is a perspective view taken from the side of an embodiment of an apparatus in accordance with the present invention.

FIG. 2 is an enlarged perspective view of a linear array housing.

FIG. 3 is an enlarged perspective view of a portion of a rotatable support of the apparatus of FIG. 1.

### DETAILED DESCRIPTION OF SPECIFIC EMBODIMENTS

As mentioned above, embodiments of the present invention are directed to apparatus comprising a rotatable support for one or more linear arrays, a mechanism for rotating the support, and a device for analyzing the results of chemical reactions. The apparatus is simple and inexpensive. The temperature of the linear arrays may be raised to a desired temperature prior to introduction of sample therein because a heater may be included on board the apparatus. The linear arrays may be examined for bubble formation subsequent to introduction of the sample. Small sample volume may be employed, which enables high sample concentration. Thus, chemical reaction time is reduced. The linear arrays may be indexed on the rotatable support using the same driving mechanism for both sample agitation and indexing. Likewise, sample removal may be carried out using the same driving mechanism. A fluid dispensing device such as, for example, a wash fluid dispenser, may be included as part of the apparatus since the apparatus provides the ability to access all entry ports of the linear arrays by indexing. In addition, the device for analyzing the results of the chemical reactions does not require moving parts to perform the examination of the linear arrays. The device is usually fixed in one position and the support with the linear arrays is rotated to present each array for examination.

The rotatable support may be any structure that provides support for the linear arrays and that is capable of rotation about an axis, usually, a central axis. The rotatable support may be, for example, a circular tray such as a carousel or the like. The circular tray and mechanism may be similar in design to that employed, for example, in the Agilent G2505 Scanner, Agilent Technologies Inc., Palo Alto Calif.

Usually, the rotatable support has a surface for receiving and holding the housings for the linear arrays. Thus, the surface of the rotatable support generally has a plurality of retaining elements for retaining the linear array housings on the surface. The design of the retaining elements is dependent on the nature of the housing for the linear array. The retaining elements should retain the housings sufficiently so that, during rotation of the support and other manipulations, the linear arrays remain securely on the surface and may be accurately examined by the analysis device to determine the results of the chemical reactions. In some embodiments, the retaining elements receive an elongated array unit in a seated position in which the retaining element extends in the radial direction while retaining the array in the seated position during rotation about an axis of the support. Such retaining elements include, for example, grooves in the surface of the rotatable support, elevated slots on the surface of the support, vacuum chuck, mechanical clamps, and so forth. The retaining elements are usually disposed around the axis of rotation of the rotatable support. The orientation of the retaining elements should be such that the housings for the linear arrays are situated so that operation of the present apparatus to move fluid through and out of the linear array may be accomplished as desired by rotation of the rotatable housing. The orientation of the retaining elements, and thus the linear arrays, should also allow accurate analysis of the reaction results.

The dimensions of the rotatable support depend on the nature of the housings of the linear arrays, the required distance of the housings from the axis of rotation of the support, the quantity of linear arrays to be processed in a single run or batch, and so forth. For linear microarray housings of approximately 3 inches in length and 0.5 inches in width, the dimensions of the rotatable support, which will house approximately 12 arrays, are usually about 8 to 12 inches in diameter. The housings for the linear arrays should be disposed from the axis of rotation of the rotatable support so that the linear array exit port is a sufficient distance from the axis of rotation so that fluid in the linear array may be moved through and out of the linear array. The housings for the linear array of the aforementioned dimensions normally are about 0.5 inches to 6 inches from the axis of rotation of the rotatable support. This is simply an example as other combinations of linear array sizes and quantities will yield different dimensions.

The number of retaining elements, and thus the number of linear array housings, on the surface of the rotatable support may be from one to many thousands or more, i.e., one to about one thousand, to about ten thousand, to about 20 thousand, and so forth.

The rotatable support is driven by a suitable driving mechanism such as a motor and the like, which is capable of rotating the rotatable support in accordance with the present invention. Accordingly, the driving mechanism should be capable of rotating the rotatable support at speeds required to overcome capillary forces in the microchannels of the linear arrays as well as rotating the rotatable support in a step-wise fashion to index the linear arrays to, for example, an examining device. A motor may be, for example, a stepping-type motor, a servo-type motor, and so forth. The driving mechanism is usually in communication with a system controller, which provides control over the direction and speed of rotation of the rotatable support, indexed movement of the rotatable support, acceleration and deceleration ramps, dwell times and so forth. The velocity profile of the rotatable support is dependent on a number of factors including, for example, the nature of the function being performed at any point in time, the assay being controlled, and so forth. For removal of fluid from the interior of the linear array, the rotatable support is rotated at a speed great enough to produce a centrifugal force that overcomes the capillary forces on the fluid. This speed is dependent on the viscosity, surface tension and so forth, of the fluid, the capillary dimensions and the surface properties of the device including material choice, surface treatments, surface roughness, designed features such as sharp edges and changes in channel dimensions, distance of the capillary from the center of rotation, the column length of the fluid along the radial path, the presence of air vents in the design, interactions of the fluids with the surface such as absorption, and the like. Thus, the speed of rotation may be, for example, about 20 to about 20,000 rpm, about 30 to about 15,000 rpm, about 50 to about 10,000 rpm.

The driving mechanism can and usually does provide for indexing of the linear arrays on the surface of the rotatable support. To assist in the indexing function, the linear array housings may comprise an identification code. A suitable reading device is employed for reading the identification code. The reading device is incorporated into the present apparatus or is separate from the present apparatus. The reading device is located so as to provide an accurate reading of the identification code of the linear array housings. The code is read at a time best suited for providing accurate identification. Thus, the reading device may read the code when each of the housings for the linear arrays is in place on the rotatable support. Alternatively, the code may also be read while the housings are loaded onto the rotatable support. Information read from the identification code is fed to a system controller for the apparatus and correlated with the indexed position of the housing on the rotatable support. In this way the identity and location of each of the linear array housings can be tracked and the processing of each linear array may be linked to the identification code. Such information may be loaded into a data storage database for use by other systems.

The identification code is usually placed in a location that does not interfere with the processing of the linear arrays. The identification code may be placed on the outside of the linear array housings. In one embodiment the present apparatus comprises a bar code reader situated on an examining device which is handheld by the operator and used when the arrays are loaded, alternatively the scanner may be mounted in a fixed position either above or below the rotatable table and each identification code is when each array is indexed to that position and so forth. Appropriate bar codes are placed on the linear array housings. The bar code may, for example, be printed on an opaque label attached to the front side, or other readable side, of the housing. Information read from the bar code can be used to identify and index the housings in an apparatus in accordance with the present invention.

In another approach a metal homing block may be employed. In one approach the homing block may be mounted on the rotatable support. The homing block then moves with the rotatable support. A metal detector may be mounted in such a manner that the movement of the rotatable support brings the homing block in the vicinity of the metal detector at some point in time during the movement of the rotatable support. A change in signal from the metal detector indicates that the homing block is in a certain position. Accordingly, during each full movement of the rotatable support, the location of a particular linear array housing is known.

The linear array housings may be moved to the present apparatus from another location manually or mechanically or a combination thereof. A linear array housing may be mechanically delivered, for example, by a robotic mechanism that involves a suitable arm or other device to move the linear array housing from a location to a retaining element of the rotatable support of the present apparatus. A portion or all of the mechanism may be part of the present apparatus or separate therefrom.

In many instances, it is desirable that the linear array housings be presented to the present apparatus in proper orientation so that accurate indexing of the housings is realized as well as accurate examination of the linear arrays for the results of chemical reactions carried out therein. Accordingly, the present apparatus or the delivery mechanism provides means to ensure the proper orientation of the linear array housings delivered to the rotatable support of the present apparatus.

The present apparatus may be designed to provide for monitoring and control of the temperature of the linear arrays in the housings on the surface of the rotatable support. The temperature of an area on the surface of the rotatable support corresponding with the area to which a linear array housing is to be delivered is heated to a predetermined temperature prior to placing the linear array housing on the rotatable support. Accordingly, the areas corresponding to the areas at which the housings are retained are subject to temperature control while the remainder of the apparatus operates without temperature control, e.g., at ambient temperature. A suitable temperature control system operates to maintain the temperature at the desired locations at optimized levels predetermined by the protocol of the chemical reactions as determined by a human operator. Alternatively, the entire apparatus is temperature controlled. The incubation of the arrays during the hybridization portion of the protocol may be quite long, typically ranging from 30 minutes to 3 days and more commonly, from a few hours to overnight. Therefore, it may be advantageous to perform the incubation process in as a batch process. The temperature control system maintains a predetermined temperature or temperatures in a range from 0 to 100 degrees C. or colder or warmer as desired.

In one embodiment a thermal source for applying heat energy above room temperature, or a thermal sink for removing heat energy below room temperature for cooling, may be employed adjacent the retaining elements. Thermal sources may apply heat energy by means of conduction from electric resistance elements, semiconductor thermo-electric heat pumps or fluid heat exchangers. Thermal sources may apply heat energy by means of radiation from long wavelength, infrared, radiation sources. Thermal sinks may remove heat energy for cooling purposes, by means of conduction from semiconductor thermoelectric heat pumps, fluid heat exchangers, compressed-gas expansion coolers, mechanical or absorption refrigeration.

The temperature may be monitored and controlled in areas other than those corresponding to the locations of the retaining elements. For example, it may be desirable to control temperature at fluid dispensing stations, at the fluid reservoirs, and the like. The temperature controlled regions may additionally comprise humidity control.

The apparatus of the present invention may optionally comprise a fluid dispensing device, which usually includes a fluid reservoir and a mechanism for delivering fluid from the reservoir to a port of the housing of the linear array. The structure of the fluid reservoir and the mechanism for delivering fluid can be of any suitable design compatible with the present apparatus. The fluid in the reservoir may be a processing fluid such as, for example, a wash solution, a fluid reagent for a chemical reaction, a buffer solution, an indicator solution such as a dye, tag or marker, and so forth. The mechanism for delivering the fluid to the housing of the linear array may be manual or automated. The latter may involve robotic technology as discussed hereinabove. Additionally, the fluid control station may comprise washable or disposable means for dispensing individual samples from a plate or tube into the linear arrays.

The fluid dispensing device generally comprises a pump for moving fluid, a suitable temperature control system that operates to control the temperature of the fluid at optimized levels, predetermined by the chemical processing protocol of choice, prior to and during fluid movement. The fluid dispensing device may additionally comprise a valve assembly, a manifold and plumbing as well as a means for metering and delivering predetermined quantities of fluid into a port of the linear array housing. Usually, the fluids to be dispensed are pumped from the dispenser, which may be held at a controlled temperature. In this regard any standard pumping technique for pumping fluids may be employed in the present apparatus. For example, pumping may be by means of a peristaltic pump, a pressurized fluid bed, a positive displacement pump, e.g., a syringe pump, and the like.

The present apparatus also comprises an examining device or detector for examining the linear array for the results of one or more chemical reactions. The nature of the examining device is dependent on the nature of the chemical reactions including any label employed for detection, such as fluorescent, chemiluminescent, colorometric based on an attached enzyme, and the like. The examining device may be a scanning device involving an imaging system or optical system. The scanning device can be any suitable apparatus for reading an array, such as one which can read the location and intensity of fluorescence at each feature of an array following exposure to a fluorescently labeled sample. Examples of examining devices that may be part of the present apparatus include Agilent G2505 Scanner, Agilent Technologies Inc., Palo Alto Calif., and the like. Other suitable apparatus and methods are described in U.S. patent applications: Ser. No. 09/846125 “Reading Multi-Featured Arrays” by Dorsel et al.; and Ser. No. 09/430214 “Interrogating Multi-Featured Arrays” by Dorsel et al. The examining device may also include a reader for reading an identification code as discussed above. For example, the examining device may comprise a reader to read bar codes appearing on the linear array housings.

The examining device is disposed with respect to the surface of the rotatable support comprising the retaining elements so that the examining device may interrogate the linear arrays retained thereon and also provide suitable indexing of the linear arrays. In one embodiment, the examining device is disposed above a surface of a rotatable support having the linear array housings retained thereon. Normally, the examining device is in a fixed position mounted on, for example, a frame member to which other components of the present apparatus are secured. As the rotatable support rotates, the linear arrays on its surface are presented to the examining device. In some embodiments the examining device is linear and oriented so as to be optically aligned with each of multiple seated array units in turn as the support is rotated. It is not necessary that the examining device be physically aligned with each of the multiple seated array units as long as it is optically aligned. However, in some embodiments physical alignment may be employed or desired.

An apparatus of the invention may include a frame to which other components of the present apparatus such as discussed above are secured. The frame may be fabricated by, for example, casting, molding, machining, and the like from laminated or reinforced metal, plastic, metal alloy, solid or aggregate stone, and the like. The frame may comprise a base member and one or more framing arms to which components of the present apparatus are attached. The base member and the framing arms may have any suitable shape such as, e.g., rectangular, circular, square, oval, and the like. The dimensions of the frame are not critical and are usually determined on a practical level depending on the shape of the frame and the number of elements thereon. The thickness of the frame should be sufficient to provide necessary stiffness to support the elements of the apparatus that are affixed to the frame. The dimensions of the frame should be sufficient to support the weight and to maintain the mechanical accuracy of the elements of the apparatus.

It may be desirable to provide a cover for the frame that will shield the elements of the apparatus. The cover for the apparatus generally permits access to components that the user must access and other elements of the apparatus usually are inaccessible to the user and typically shielded from view. To this end, a portion of the cover of the apparatus may be opaque so that the user is not able to view these elements. It is often desirable that light not be permitted to enter an apparatus of the invention because certain labels used in, for example, the polynucleotide detection systems discussed herein are light sensitive. Accordingly, shielding of certain areas within the interior of an apparatus is desirable and may be accomplished, for example, with a cover that does not allow light to enter the areas of concern. In some circumstances, the cover will not permit light to enter any part of the covered portion of the apparatus. Of course, the elements of an apparatus should be accessible for repair and maintenance normally by a trained technician.

An apparatus of the invention may also include appropriate electrical and mechanical architecture for operation of the various elements of the apparatus and provide for the various components of the apparatus to operate in concert. In this way linear array housings may be placed on the surface of a rotatable support, sample may be delivered to ports in the linear array housings, the rotatable support may be rotated to appropriate speeds, processing fluid may be moved to ports in the linear array housings or collected from ports, the examining device may be activated to examine the linear arrays, and so forth. Such architecture includes, for example, electrical sensors, electromechanical actuators and motors, temperature and motion control electronic circuitry, operator controls and displays, electrical wiring and connections, fluid and pneumatic sensors, actuators, valves and pumps, fluid and gas tubing and plumbing, fluid control electronic circuitry bearings, load sensors, etc., and so forth. The apparatus may also comprise various computer interfaces or processors for controlling the various components of the present apparatus. For example, embodiments of an apparatus may comprise a processor that controls rotation of the support such that at one speed fluid is removed from the linear array and at another speed the support is advanced and held for examination by the examining device.

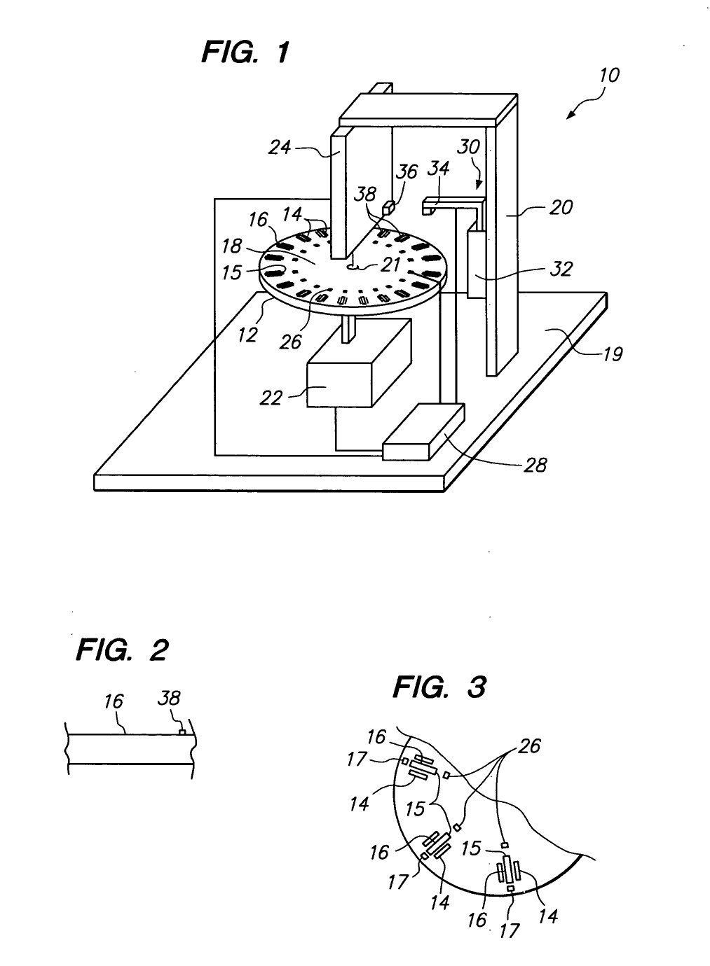

An example of an apparatus in accordance with the present invention is depicted in FIGS. 1-3. Apparatus 10 comprises a circular tray 12 having a plurality of retaining elements 14, which retain linear array housings 16 on surface 18 of circular tray 12. Circular tray 12 is rotatably mounted on base member 19 of frame 20 and driven by motor 22 about axis 21. Apparatus 10 further comprises scanning device 24, which is fixedly attached to frame 20. Heaters 26 are adjacent to retaining elements 14 and communicate with computer 28, with which motor 22 and scanning device 24 also communicate. Apparatus 10 also comprises fluid dispensing device 30, which comprises fluid reservoir 32 and robotic arm 34. Fluid dispensing device 30 also communicates with computer 28. Apparatus 10 also comprises reading element 36 for reading an identifier 38 on the housing of linear arrays 16. Reading element 36 also communicates with computer 28.

The present apparatus are designed for use with linear arrays. As mentioned above, a linear array may be a one-dimensional array of features bound in a non-diffusive manner to a surface. By the term “non-diffusive” is meant that the molecules that make up the individual features are bound to the surface in such a manner that they will not detach under the conditions of preparing and using the linear array. Non-diffusive binding may be covalent or may be non-covalent or macromolecular association where the linking is of sufficient strength to withstand the aforementioned conditions. Non-diffusive binding of the features may be achieved in a number of approaches known in the art. Some of those approaches are discussed briefly hereinbelow by way of illustration and not limitation.

The features generally are molecules that are involved in chemical reactions such as, for example, the detection of target molecules or analytes in a sample of interest. Each molecule of a feature may be specific for a corresponding analyte or for a compound indicative of the presence of the analyte. For example, the analyte may be part of a complex such as, for example, an antigen-antibody complex, polynucleotide-protein complex, polynucleotide-polynucleotide complex and the like, and the feature is capable of binding to a component of the complex. Usually, the molecule comprising the feature is a specific binding partner for the analyte or for a member of the complex indicative of the presence of the analyte.

Each feature, or element, within the linear array is defined to be a small, regularly shaped region of the surface of the substrate. The features in the linear array are arranged in a predetermined manner. Each feature of a linear array usually carries a predetermined chemical compound or mixtures thereof and is typically of homogeneous composition. Each feature within the linear array may contain a different molecular species, and the molecular species within a given feature may differ from the molecular species within the remaining features of the molecular array. Some or all of the features may be of different compositions. Each array may be separated by spaces or areas. Interarray areas and interfeature areas are usually present but are not essential. These interarray and interfeature areas do not carry any chemical compound such as polynucleotide (or other biopolymer of a type of which the features are composed). Interarray areas and interfeature areas typically will be present where arrays are formed by the conventional in situ process or by deposition of previously obtained moieties, as described herein, by depositing for each feature at least one droplet of reagent such as from a pulse jet but may not be present when, for example, photolithographic array fabrication processes are used. It will be appreciated though that the interarray areas and interfeature areas, when present, could be of various sizes and configurations.

In the linear array the order of the features identifies each feature, which allows selective identification of target molecules. Usually, the linear array has a fixed length determined by the number of features of the linear array. The linear array is elongated, i.e., the linear array comprises rows of features where the rows generally have more features along their length than the number of rows of the array. In general, the linear arrays have a length to width in terms of numbers of features of at least about 10 to 1, about 20 to 1, about 30 to 1, about 40 to 1, about 50 to 1, about 100 to 1, about 500 to 1, about 1000 to 1, about 10,000 to 1, about 100,000 to 1 and so forth.

In some embodiments the width of the linear array is one feature. However, for purposes of the present invention, the width of the linear array may be greater than one feature where the size of the feature and the width of the housing, e.g., microchannel, permit. Therefore, in certain embodiments the width of the linear array may be 1 to about 5 features, 1 to about 4 features, 1 to about 3 features, 1 to 2 features. In such an embodiment where the linear array is more than one feature wide, each feature comprising the width at the position in question is the same or different and each feature comprising the length of the linear array may be the same or different, usually different, as discussed above. In certain embodiments the width of the linear array may be greater than 5 features. The width of the features, for example, the diameter of a round spot, may be in the range from about 10 μm to about 1.0 cm. In other embodiments each feature may have a width in the range of about 1.0 μm to about 1.0 mm, usually about 5.0 μm to about 500 μm, and more usually about 10 μm to about 200 μm. Non-round features may have width ranges equivalent to that of circular features with the foregoing width (diameter) ranges.

The linear array is generally present in a channel that permits capillary forces to act upon fluid in or around the channel. The channel may be partially or fully enclosed as long as such capillary forces may be realized. The channel may be in a housing, which may be a microchannel. The housing is any enclosure in which at least a portion of the interior has capillary dimensions. In one approach, the microchannel is part of a microfluidic system. Microfluidic systems have been developed for performing chemical, clinical, and environmental analysis of chemical and biological specimens. The term microfluidic system refers to a system or device having a network of chambers connected by channels, in which the channels have microscale features, that is, features too small to examine with the unaided eye. The channel often has a capillary dimension, i.e., a cross-sectional area that provides for capillary flow through the channel. At least one of the cross-sectional dimensions, e.g., width, height, diameter, is at least about 1 μm, usually at least about 10 μm, and is usually no more than about 500 μm, or no more than about 200 μm. Channels of capillary dimension typically have an inside bore diameter (ID) of from about 1 to about 200 microns, more typically from about 25 to about 100 microns. The term “microfluidic” generally means “of or pertaining to fluids and being of a magnitude on the order consistent with capillary dimension.” The channel(s) may be part of a microfluidic network or a system of interconnected cavity structures and capillary-size channels configured with a plurality of branches through which fluids may be manipulated and processed. However, in one form, the present devices comprise a microchannel in the form of a tube within a housing.

Such microfluidic systems are often fabricated using photolithography, wet chemical etching, and other techniques similar to those employed in the semiconductor industry. The resulting devices can be used to perform a variety of sophisticated chemical and biological analytical techniques.

The channel is thus a conduit by which a sample may contact a linear array. The channels, and thus the linear array, may be straight, curved, serpentine, labyrinth-like or other convenient configuration comprised of separate tubes or part of a monolithic, often planar, substrate. The cross-sectional shape of the channel is not critical and may be circular, ellipsoid, square, rectangular, triangular and the like. The inside of the channel may be coated with a material for strength, for enhancing or reducing electrokinetic flow, for enhancing detection limits and sensitivity, and so forth. Exemplary of coatings are silylation, polyacrylamide (vinyl bound), methylcellulose, polyether, polyvinylpyrrolidone, and polyethylene glycol, polypropylene, Teflon™ (DuPont), Nafion™ (DuPont), and the like may also be used.

The channel usually comprises at least one entry port, namely, any site at which a liquid may be introduced into a device having one or more channels. The entry port may be a well or simply the terminus of a channel that opens any place on the device such as at an edge. The channel usually comprises at least one port from which fluid exiting the channel may travel to a collection chamber or the like, which is in fluid communication with the channel.

Moving materials through microchannels may be accomplished, for example, by use of a fluid pressure difference, by use of various electro-kinetic processes including electrophoresis, electroosmotic flow, and electrokinetic pumping, and so forth. Microfluidic devices generally include one or more channels fabricated on or within the devices, usually within the devices. The devices also can include reservoirs, fluidly connected to the channels, which can be used, for example, to introduce materials into the channels to contact a linear array contained in the channel, to receive and store waste fluids such as removed sample, wash solutions, labeling solutions, etc., to more precisely control the reaction or to provide some or all of the reagents needed for the reaction with the device rather than supplying them in bulk or in a separate kit and so forth.

Microfluidic systems have a number of advantages over conventional chemical or physical laboratory techniques. For example, microfluidic systems are particularly well adapted for analyzing small sample sizes, typically making use of samples on the order of nanoliters and even picoliters. The substrates may be produced at relatively low cost, and the channels can be arranged to perform numerous specific analytical operations, including mixing, dispensing, valving, reactions, detections, electrophoresis, and the like. The analytical capabilities of such microfluidic systems may be enhanced by increasing the number and complexity of network channels, reaction chambers, and the like.

As mentioned above, the length of the linear array as manufactured is usually a fixed length determined by the number of features of the linear array. The number of features is related to the nature of the features, the nature of the analytes, the biological questions being asked, the sensitivity or specificity required, and so forth. A typical linear array may contain more than about ten, more than about one hundred, more than about one thousand, more than about ten thousand, more than about twenty thousand, etc., more than about one hundred thousand, features and so forth.

The housing for the linear array in accordance with the present invention may be any suitable housing for a microfluidic system. The housing comprising the linear array may be provided in any number of convenient forms depending on the nature of the housing, the nature of the linear array, the nature of the assay, manufacturing or use requirements, and so forth. The material for the housing should provide physical support for the chemical compounds that are deposited on an interior surface of the housing or synthesized on an interior surface of the housing in situ from subunits. The materials should be of such a composition that they endure the conditions of a deposition process and/or an in situ synthesis and of any subsequent treatment or handling or processing that may be encountered in the use of a particular array.

Typically, the housing material is transparent or comprises a viewing area that is transparent. By “transparent” is meant that the substrate material permits signal from features on an interior surface of the substrate to pass therethrough without substantial attenuation and also permits any interrogating radiation to pass therethrough without substantial attenuation. By “without substantial attenuation” may include, for example, without a loss of more than about 40% or without a loss of more than about 30%, about 20% or about 10%, of signal. The interrogating radiation and signal may for example be visible, ultraviolet or infrared light. In certain embodiments, such as for example where production of binding pair arrays for use in research and related applications is desired, the materials from which the substrate may be fabricated should ideally exhibit a low level of non-specific binding during hybridization events.

The materials may be naturally occurring or synthetic or modified naturally occurring. Particular plastics finding use include, for example, polyethylene, polypropylene, polytetrafluoroethylene (PTFE), e.g., TEFLON®, polymethylmethacrylate, polycarbonate, polyethylene terephthalate, polystyrene or styrene copolymers, polyurethanes, polyesters, polycarbonates, polyureas, polyamides, polyethyleneamines, polyarylene sulfides, polysiloxanes, polydimethylsiloxanes, polyimides, polyacetates, poly etheretherketone (PEEK), and the like, either used alone or in conjunction with another material or materials. The housing may be rigid or flexible.

Suitable rigid materials may include glass, which term is used to include silica, and include, for example, glass such as glass available as Bioglass, and suitable rigid plastics and resins, and so forth. Rigid plastics include, for example, polymers such as, e.g., poly (vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc., either used by themselves or in conjunction with other materials.

The housing comprising the linear array may be prepared in a number of ways. The following discussion is by way of illustration and not limitation. In one approach, the linear array is synthesized or deposited on the surface of a housing substrate and the area comprising at least the linear array is enclosed to form a channel comprising the linear array. Enclosure may be attained using an appropriate material to cover the channel and then sealing to form the housing. The apparatus may be fabricated using other convenient means, including conventional molding and casting techniques, extrusion sheet forming, calendaring, thermoforming, and the like. For example, with apparatus prepared from a plastic material, a silica mold master, which is negative for the network structure in the planar substrate of one plate can be prepared by etching or laser micromachining. In addition to having a raised ridge, which forms the channel in the substrate, the silica mold may have a raised area that provides for one or more cavity structures in the planar substrate. Next, a polymer precursor formulation can be thermally cured or photopolymerized between the silica master and support planar plate, such as a glass plate.

In one embodiment, the linear array may be synthesized or deposited on the surface of a flexible material or substrate in the dimensions desired. For example, for a microarray the chemical compounds comprising the linear array are synthesized or deposited in an area that corresponds to capillary dimensions. The flexible substrate may be substantially flat along the area of synthesis or deposition or there may be a groove, depression, or the like in the housing substrate where the linear array is placed. This area of deposition or synthesis is ultimately enclosed to form a channel having the linear array therein. See, for example, U.S. patent application Ser. No. 10/037757, entitled “Chemical Arrays” by Schembri, et al., filed Oct. 18, 2001, published as U.S. Patent Publication No. 20030108726 and U.S. patent application Ser. No. 10/032608, entitled “Chemical Arrays”, by Lefkowitz, et al., filed Oct. 18, 2001, published as U.S. Patent Publication No. 20030077380, the disclosures of which are incorporated herein by reference.

Enclosing the housing to form the channel comprising the linear array may be accomplished in a number of ways. One important consideration in forming the linear array housing in general, and enclosing the housing in particular, is to avoid damage to the linear array on the surface of the housing substrate. In one approach, for example, the substrate is a flexible material that is folded over to enclose the housing to form the channel. After folding, the flexible material is sealed to itself in an area outside the area of the channel. Sealing may be achieved by application of heat, adhesives, and so forth.

In an alternate approach, a separate material may be placed over the substrate comprising the linear array and sealed to the substrate to enclose the housing to form the channel with the linear array therein. The separate material may be sealed to the substrate as discussed above. The separate material may have the same composition as the substrate or a composition that is different from the substrate. A primary consideration is that the separate material is severable as discussed above.

The interior surface of the housing to which a plurality of chemical compounds is attached to form the linear array can be hydrophilic or capable of being rendered hydrophilic or it may be hydrophobic. The interior surface is normally treated to create a primed or functionalized surface, that is, a surface that is able to support the attachment of a fully formed chemical compound or the synthetic steps involved in the production of the chemical compound on the surface of the substrate. Functionalization relates to modification of the surface of a substrate to provide a plurality of functional groups on the substrate surface. By the term “functionalized surface” is meant a substrate surface that has been modified so that a plurality of functional groups are present thereon usually at discrete sites on the surface. The manner of treatment is dependent on the nature of the chemical compound to be synthesized or deposited and on the nature of the surface. In one approach a reactive hydrophilic site or reactive hydrophilic group is introduced onto the surface of the substrate. Such hydrophilic moieties can be used as the starting point in a synthetic organic process.

The surface of the housing onto which the chemical compounds are deposited or formed may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethylene amines, polyarylene sulfides, polysiloxanes, polyimides, polyacetates, and the like, where the polymers may be hetero- or homo-polymeric, and may or may not have separate functional moieties attached thereto (for example, conjugated). Various further modifications to the particular embodiments described above are, of course, possible. Accordingly, the present invention is not limited to the particular embodiments described in detail above.

As mentioned above, the chemical compounds that are bound to the interior surface of the housing to form the linear array may be synthesized or deposited on the surface. Usually, an initial derivatization of the surface is carried out. Modification of surfaces for use in chemical synthesis has been described. See, for example, U.S. Pat. No. 5,266,222 (Willis) and U.S. Pat. No. 5,137,765 (Farnsworth).

The arrays may be, and usually are, microarrays created on the interior surface of the housing by in situ synthesis of biopolymers such as polynucleotides, polypeptides, polysaccharides, etc., and combinations thereof, or by deposition of molecules such as oligonucleotides, cDNA and so forth. In general, arrays are synthesized on a surface by one of any number of synthetic techniques that are known in the art.

In one embodiment, the surface of the substrate is siliceous, i.e., the surface comprises silicon oxide groups, either present in the natural state or introduced by techniques well known in the art. One technique for introducing siloxyl groups onto the surface involves reactive hydrophilic moieties on the surface. These moieties are typically epoxide groups, carboxyl groups, thiol groups, and/or substituted or unsubstituted amino groups as well as a functionality that may be used to introduce such a group such as, for example, an olefin that may be converted to a hydroxyl group by means well known in the art. One approach is disclosed in U.S. Pat. No. 5,474,796 (Brennan), the relevant portions of which are incorporated herein by reference. A siliceous surface may be used to form silyl linkages, i.e., linkages that involve silicon atoms. Usually, the silyl linkage involves a silicon-oxygen bond, a silicon-halogen bond, a silicon-nitrogen bond, or a silicon-carbon bond.

Another method for attachment is described in U.S. Pat. No. 6,219,674 (Fulcrand, et al.). A surface is employed that comprises a linking group consisting of a first portion comprising a hydrocarbon chain, optionally substituted, and a second portion comprising an alkylene oxide or an alkylene imine wherein the alkylene is optionally substituted. One end of the first portion is attached to the surface and one end of the second portion is attached to the other end of the first portion chain by means of an amine or an oxy functionality. The second portion terminates in an amine or a hydroxy functionality. The surface is reacted with the substance to be immobilized under conditions for attachment of the substance to the surface by means of the linking group.

Another method for attachment is described in U.S. Pat. No. 6,258,454 (Lefkowitz, et al.). A solid substrate having hydrophilic moieties on its surface is treated with a derivatizing composition containing a mixture of silanes. A first silane provides the desired reduction in surface energy, while the second silane enables functionalization with molecular moieties of interest, such as small molecules, initial monomers to be used in the solid phase synthesis of oligomers, or intact oligomers. Molecular moieties of interest may be attached through cleavable sites.

A procedure for the derivatization of a metal oxide surface uses an aminoalkyl silane derivative, e.g., trialkoxy 3-aminopropylsilane such as aminopropyltriethoxy silane (APS), 4-aminobutyltrimethoxysilane, 4-aminobutyltriethoxysilane, 2-aminoethyltriethoxysilane, and the like. APS reacts readily with the oxide and/or siloxyl groups on metal and silicon surfaces. APS provides primary amine groups that may be used to carry out the present methods. Such a derivatization procedure is described in EP 0 173 356 B1, the relevant portions of which are incorporated herein by reference. Other methods for treating the surface of a substrate to which the chemical compounds become bound will be suggested to those skilled in the art in view of the teaching herein.

The invention has particular application to linear arrays of oligomers or polymers. The oligomer or polymer is a chemical entity that contains a plurality of monomers. It is generally accepted that the term “oligomers” is used to refer to a species of polymers. The terms “oligomer” and “polymer” may be used interchangeably herein. Polymers usually comprise at least two monomers. Oligomers generally comprise about 6 to about 20,000 monomers, typically, about 10 to about 10,000, more typically about 15 to about 4,000 monomers. Examples of polymers include polydeoxyribonucleotides, polyribonucleotides, other polynucleotides that are C-glycosides of a purine or pyrimidine base, or other modified polynucleotides, polypeptides, polysaccharides, and other chemical entities that contain repeating units of like chemical structure. Exemplary of oligomers are oligonucleotides and peptides.

A monomer is a chemical entity that can be covalently linked to one or more other such entities to form an oligomer or polymer. Examples of monomers include nucleotides, amino acids, saccharides, peptoids, and the like and subunits comprising nucleotides, amino acids, saccharides, peptoids and the like. The subunits may comprise all of the same component such as, for example, all of the same nucleotide or amino acid, or the subunit may comprise different components such as, for example, different nucleotides or different amino acids. The subunits may comprise about 2 to about 2000, or about 5 to about 200, monomer units. In general, the monomers have first and second sites (e.g., C-termini and N-termini, or 5′ and 3′ sites) suitable for binding of other like monomers by means of standard chemical reactions (e.g., condensation, nucleophilic displacement of a leaving group, or the like), and a diverse element that distinguishes a particular monomer from a different monomer of the same type (e.g., an amino acid side chain, a nucleotide base, etc.). The initial substrate-bound, or support-bound, monomer is generally used as a building block in a multi-step synthesis procedure to form a complete ligand, such as in the synthesis of oligonucleotides, oligopeptides, oligosaccharides, etc. and the like.

A biomonomer references a single unit, which can be linked with the same or other biomonomers to form a biopolymer (for example, a single amino acid or nucleotide with two linking groups one or both of which may have removable protecting groups). A biomonomer fluid or biopolymer fluid references a liquid containing either a biomonomer or biopolymer, respectively (typically in solution).

A biopolymer is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions.

Polynucleotides are compounds or compositions that are polymeric nucleotides or nucleic acid polymers. The polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. The polynucleotide can have from about 2 to 5,000,000 or more nucleotides. Usually, the oligonucleotides are at least about 2 nucleotides, usually, about 5 to about 100 nucleotides, more usually, about 10 to about 50 nucleotides, and may be about 15 to about 30 nucleotides, in length. Polynucleotides include single or multiple stranded configurations, where one or more of the strands may or may not be completely aligned with another.

A nucleotide refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “polynucleotide” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides.

As mentioned above, biopolymer arrays can be fabricated by depositing previously obtained biopolymers (such as from synthesis or natural sources) onto a substrate, or by in situ synthesis methods. The in situ synthesis methods include those described in U.S. Pat. No. 5,449,754 for synthesizing peptide arrays, as well as WO 98/41531 and the references cited therein for synthesizing polynucleotides (specifically, DNA). Such in situ synthesis methods can be basically regarded as repeating at each spot the sequence of: (a) deprotecting any previously deposited monomer so that it can now link with a subsequently deposited protected monomer; and (b) depositing a droplet of another protected monomer for linking. Different monomers may be deposited at different regions on the substrate during any one iteration so that the different regions of the completed array will have different desired biopolymer sequences. One or more intermediate further steps may be required in each iteration, such as oxidation, capping and washing steps. The deposition methods basically involve depositing biopolymers at predetermined locations on a substrate, which are suitably activated such that the biopolymers can link thereto. Biopolymers of different sequence may be deposited at different regions of the substrate to yield the completed array. Washing or other additional steps may also be used. Reagents used in typical in situ synthesis are water sensitive, and thus the presence of moisture should be eliminated or at least minimized.

The in situ method for fabricating a polynucleotide array typically follows, at each of the multiple different addresses at which features are to be formed, the same conventional iterative sequence used in forming polynucleotides from nucleoside reagents on a substrate by means of known chemistry. This iterative sequence is as follows: (a) coupling a selected nucleoside through a phosphite linkage to a functionalized substrate in the first iteration, or a nucleoside bound to the substrate (i.e. the nucleoside-modified substrate) in subsequent iterations; (b) optionally, but typically, blocking unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The functionalized substrate (in the first cycle) or deprotected coupled nucleoside (in subsequent cycles) provides a substrate bound moiety with a linking group for forming the phosphite linkage with a next nucleoside to be coupled in step (a). A number of reagents involved in the above synthetic steps such as, for example, phosphoramidite reagents, are sensitive to moisture and anhydrous conditions and solvents are employed. Final deprotection of nucleoside bases can be accomplished using alkaline conditions such as ammonium hydroxide, in a known manner.

The foregoing chemistry of the synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, and 5,869,643, EP 0294196, and elsewhere.

As mentioned above, various ways may be employed to produce an array of polynucleotides on the surface of a substrate. Such methods are known in the art. One in situ method employs pulse-jet technology to dispense the appropriate phosphoramidite reagents and other reagents onto individual sites on a surface of a substrate. Oligonucleotides are synthesized on a surface of a substrate in situ using phosphoramidite chemistry. Solutions containing nucleotide monomers and other reagents as necessary such as an activator, e.g., tetrazole, are applied to the surface of a substrate by means of thermal pulse-jet technology (although piezoelectric activated pulse jets might also be used, but in any event the pulse jets used must be constructed of materials chemically compatible with the solutions used). Individual droplets of reagents are applied to reactive areas on the surface using, for example, a thermal pulse-jet type nozzle. The surface of the substrate may have an alkyl bromide trichlorosilane coating to which is attached polyethylene glycol to provide terminal hydroxyl groups. These hydroxyl groups provide for linking to a terminal primary amine group on a monomeric reagent. Excess of non-reacted chemical on the surface is washed away in a subsequent step. For example, see U.S. Pat. No. 5,700,637 and PCT WO 95/25116 and PCT application WO 89/10977.

Another approach for fabricating an array of biopolymers on a substrate using a biopolymer or biomonomer fluid and using a fluid dispensing head is described in U.S. Pat. No. 6,242,266 (Schleifer, et al.). The head has at least one jet that can dispense droplets onto a surface of a substrate. The jet includes a chamber with an orifice and an ejector, which, when activated, causes a droplet to be ejected from the orifice. Multiple droplets of the biopolymer or biomonomer fluid are dispensed from the head orifice so as to form an array of droplets on the surface of the substrate.

In another embodiment (U.S. Pat. No. 6,232,072) (Fisher) a method of, and apparatus for, fabricating a biopolymer array is disclosed. Droplets of fluid carrying the biopolymer or biomonomer are deposited onto a front side of a transparent substrate. Light is directed through the substrate from the front side, back through a substrate backside and a first set of deposited droplets on the first side to an image sensor.

An example of another method for chemical array fabrication is described in U.S. Pat. No. 6,180,351 (Cattell). The method includes receiving from a remote station information on a layout of the array and an associated first identifier. A local identifier is generated corresponding to the first identifier and associated array. The local identifier is shorter in length than the corresponding first identifier. The addressable array is fabricated on the substrate in accordance with the received layout information.

As indicated above, in the present invention the housing for the linear array or the linear array itself may be identified by some marking so that the various arrays placed on the present apparatus may be indexed. Such markings may be, for example, bar codes, and the like. The markings may be applied to the exterior of the housing by a suitable writing system, which is under the control of a processor. The writing system also includes a writer in the form of a printer that applies markings onto the exterior of the housing by printing them in the form of, for example, bar codes, directly onto the housing of the substrate (or indirectly such as onto a label later attached to the substrate). Each marking is associated with a corresponding array. In this context “printing” is used to include any appropriate means of applying the markings, such as by ink, laser ablation, impressing, and the like.

The markings may include an identifier, which is generated and used as described in U.S. Pat. No. 6,180,351 titled “Chemical Array Fabrication with Identifier”. The identifiers may also optionally include a communication address that identifies the address of a remote location on communication channel from which one or more characteristics of an array will be communicated in response to a received communication of the associated identifier. Such remote location may be that of communication module or alternatively that of another accessible memory on a communication channel carrying the database of array characteristic data and associated identifiers. Examples of a communication address may be a telephone number, computer ID on a WAN, or an internet Universal Resource Locator.

In a method for conducting chemical reactions in accordance with the present invention, a sample is introduced into a housing by capillary action wherein the chemical reactions are conducted within the housing. The sample is incubated in the housing under conditions for carrying out the chemical reactions. The housing is then rotated to remove the sample from the housing.

An apparatus in accordance with the present invention may be employed to conduct the chemical reactions in the linear arrays. In one approach various assays involving biopolymers may be carried out. Usually, a plurality of linear array housings is secured on the surface of a rotatable support by means of retaining elements. The linear arrays may be heated to a predetermined temperature using on-board heating elements. An entry port of the housing for each of the linear arrays is exposed to a sample suspected of containing the analyte(s) of interest (for example, a fluorescent-labeled polynucleotide or protein-containing sample). The sample may be introduced manually or mechanically. For example, a user may place the sample at the entry port by suitable means such as a pipette or the like. Alternatively, a computer controlled robotic system can be employed. A computer controls the location of the application of the sample so that the sample is placed in the correct channel or housing. An indexing system as discussed above assists in this process.

Other means of introducing the sample to the linear array include, for example, a sipper structure as disclosed in U.S. Patent Application Publication No. 20030070833 (Barth.), the relevant disclosure of which is incorporated herein by reference.

In another approach an external valve may control the sample movement. See, for example, U.S. Pat. No. 6,194,900 (Freeman), which describes a microfluidic circuit with valves to perform a biological assay.

The sample is usually introduced into the microchannel of the housing device through an opening that corresponds to the beginning and is moved into the microchannel by capillary action or capillary force. However, it is within the purview of the invention to assist movement of the sample into the channel. Approaches are known to those skilled in the art such as, for example, centrifugal force below the level required to empty the channel though the output, pressure assist, vacuum assist, and so forth.

The linear array may be scanned to assure that no bubbles are trapped in the microchannel. To this end, the array is scanned and the reflected light is detected. The scanner is looking for sudden changes in reflectivity indicating that a meniscus or bubble interface has been detected. The linear array chamber may then be automatically emptied and refilled to remove the bubble. Alternatively, additional sample is pulled into the linear array to drive the bubble out of the active area.

After the linear array has been contacted with the sample, the sample and the linear array are maintained in contact for a period of time and under conditions sufficient to achieve the desired chemical reactions. The conditions for a reaction, such as, for example, period of time of contact, temperature, pH, salt concentration and so forth, are dependent on the nature of the chemical reaction, the nature of the chemical reactants including the liquid samples, and the like. Conditions for binding of members of specific binding pairs are generally well known and will not be discussed in detail here. The conditions for the various processing steps are also known in the art.

The time and conditions depend on the nature of the chemical reactions and, generally, will be apparent to those skilled in the art in view of the disclosure herein and the knowledge of the skilled artisan. One or more incubation periods may be employed. It may be desirable to agitate the sample in the contact with the linear array to promote the chemical reactions. Agitation may be achieved by any suitable means such as, for example, rocking, centrifugal force, capillary valve, acoustics, and the like.

It is desirable to keep the linear array from drying. Thus, the linear array may be maintained wet at all times by contact with sample or other fluid such as wash solution and the like. In many instances residual sample and other fluids will accomplish this wetting. Drying of the linear arrays is dependent on a number of factors such as, for example, the speed at which the sample and other fluids traverse the linear array, the hydrophobicity of the surface, the surface tension and viscosity of the fluid, the presence of surfactants, and so forth. Ideally, the linear array surface is kept uniformly wet, i.e., the surface has a film of fluid that has uniform thickness and so forth.

After the desired incubation period(s), the sample is removed from the microchannel. The rotatable support on which the linear array housings are disposed is rotated at a speed and for a time sufficient to overcome capillary forces within the microchannel and move the sample out of the microchannel. For this purpose, the speed of rotation is dependent on the fluid properties, the capillary dimensions, the surface properties, the distance of the output from the center of rotation, the geometry of the opening, the presence of a vent, and the like. The speed of rotation for this aspect of the invention may be determined empirically based on the disclosure herein.

Sample may be removed through a port of the microchannel. The port may be an exit port separate from the entry port or it may be the entry port itself under certain circumstances. The port from which the sample and other fluids exit the microchannel is in fluid communication with a chamber for containing the removed materials. Desirably, the chamber is within the linear array housing. There may be one or more chambers for containing waste sample and fluids. The size of the chambers is dependent on the volume of the waste sample and fluids. The volume of the chambers should be at least sufficient to contain all of the materials directed to the chambers. Such chambers are well-known in the art and will not be discussed further.

Either following removal of the sample or in conjunction therewith, wash fluid is introduced into the microchannel in a manner similar to that for the sample itself. In one approach the wash fluid may be employed to push sample out of the microchannel. The amount of wash fluid is dependent on the nature of the sample, the nature of the chemical reactions, the size of the microchannel, the stringency of the wash fluid to ensure that non-specifically bound material is released from the surface or probes, and so forth. Multiple washings may be employed. Only one wash fluid dispenser may be used (although more could be present) since the entry ports of the linear arrays are on the same rotational axis. The wash fluid is usually agitated in the microchannel in a manner similar to that for the sample.

The wash fluid is maintained in the microchannel for a period of time sufficient to accomplish removal of sample from the microchannel. The amount of time is dependent on the nature of the sample and the surface bound probes, the surface properties and the like, and is usually about 2 seconds to about 30 minutes, about 5 seconds to about 20 minutes. Wash fluid is removed from the microchannel to a collection chamber in a manner similar to that discussed above for removal of the sample. There may be additional wash steps and the addition of a dye or tag followed by more washes. Depending on the nature of the detection step, the array may be scanner wet or dry. Wash fluid is removed from the microchannel to a collection chamber in a manner similar to that discussed above for removal of the sample.

Following incubation and wash steps, the linear array is examined for the results of the chemical reactions. Each array is indexed to an examining device by rotation of the rotatable support. The examination process and device are dependent on a number of factors such as, for example, the nature of the chemical reactions, the nature of detectable moieties employed, and so forth. The examining device may be associated directly with the rotatable support. The examining device is fixed with respect to the rotatable support so that no moving parts are required for the scan except for rotation of the rotatable support.

A method of the invention will be described next with reference to FIG. 1. Linear array housings 16 are placed on surface 18 of circular tray 12 in retaining elements 14. The temperature of the linear arrays is raised to a predetermined level by activating heaters 26. Sample is placed at entry ports 15 of each of housings 16 and enters the linear arrays drawn by capillary forces of the microchannel. Optionally, the linear array may be scanned for the presence of bubbles by the detection means. It should be noted that the sample could be placed, alternatively, at a port of housing 16 that is opposite to port 15. In either situation, sample would be drawn into housing 16 by means of capillary action. After a predetermined length of time for incubation, circular tray 12 is rotated by activation of motor 22 to a speed sufficient to move sample out of linear arrays 16 and into reservoirs 17. Each of reservoirs 17 is in fluid communication with housings 16 and has a fluid capacity to receive and retain residual sample, wash solutions and so forth. As mentioned above, wash fluid may be employed to push sample out of linear array 16. To this end, wash fluid is dispensed to each linear array 16 by means of robotic arm 34 and drawn into the array by capillary action. This wash fluid is removed by centrifugal force. After sufficient washing of the linear array surface, motor 22 is activated to index each linear array housing to scanning device 24, which examines the linear arrays for the results of the chemical reactions. Indexing is assisted by reader 36, which reads identification codes 38 on linear array housings 16. The apparatus is controlled by computer 28, which also serves to collect and store the data representing the results of the chemical reactions.

The sample may be a trial sample, a reference sample, a combination of the foregoing, or a known mixture of components such as polynucleotides, proteins, polysaccharides and the like (in which case the arrays may be composed of features that are unknown such as polynucleotide sequences to be evaluated). The samples may be from biological assays such as in the identification of drug targets, single-nucleotide polymorphism mapping, monitoring samples from patients to track their response to treatment and/or assess the efficacy of new treatments, and so forth. For hybridization reactions the sample generally comprises a target molecule that may or may not hybridize to a surface-bound molecular probe. The term “target molecule” refers to a known or unknown molecule in a sample, which will hybridize to a molecular probe on a substrate surface if the target molecule and the molecular probe contain complementary regions. In general, the target molecule is a “biopolymer,” i.e., an oligomer or polymer. The present devices and methods have particular application to various processing steps involved with the aforementioned hybridization reactions.

An oligonucleotide probe may be, or may be capable of being, labeled with a reporter group, which generates a signal, or may be, or may be capable of becoming, bound to one a feature of the linear array. Detection of signal depends upon the nature of the label or reporter group. Commonly, binding of an oligonucleotide probe to a target polynucleotide sequence is detected by means of a label incorporated into the target. Alternatively, the target polynucleotide sequence may be unlabeled and a second oligonucleotide probe may be labeled. Binding can be detected by separating the bound second oligonucleotide probe or target polynucleotide from the free second oligonucleotide probe or target polynucleotide and detecting the label. In one approach, a sandwich is formed comprised of one oligonucleotide probe, which may be labeled, the target polynucleotide and an oligonucleotide probe that is or can become bound to a surface of a support. Alternatively, binding can be detected by a change in the signal-producing properties of the label upon binding, such as a change in the emission efficiency of a fluorescent or chemiluminescent label. This permits detection to be carried out without a separation step. Finally, binding can be detected by labeling the target polynucleotide, allowing the target polynucleotide to hybridize to a surface-bound oligonucleotide probe, washing away the unbound target polynucleotide and detecting the labeled target polynucleotide that remains. Direct detection of labeled target polynucleotide hybridized to surface-bound oligonucleotide probes is particularly advantageous in the use of ordered arrays.

As mentioned above, in one approach cell matter is lysed, to release its DNA as fragments, which are then separated out by electrophoresis or other means, and then tagged with a fluorescent or other label. The DNA mix is exposed to the linear array of oligonucleotide probes, whereupon selective attachment to matching probe sites takes place. The array is then washed and the result of exposure to the array is determined. In this particular example, the array is imaged by scanning the surface of the support so as to reveal for analysis and interpretation the sites where attachment occurred.

The signal referred to above may arise from any moiety that may be incorporated into a molecule such as an oligonucleotide probe for the purpose of detection. Often, a label is employed, which may be a member of a signal producing system. The label is capable of being detected directly or indirectly. In general, any reporter molecule that is detectable can be a label. Labels include, for example, (i) reporter molecules that can be detected directly by virtue of generating a signal, (ii) specific binding pair members that may be detected indirectly by subsequent binding to a cognate that contains a reporter molecule, (iii) mass tags detectable by mass spectrometry, (iv) oligonucleotide primers that can provide a template for amplification or ligation and (v) a specific polynucleotide sequence or recognition sequence that can act as a ligand such as for a repressor protein, wherein in the latter two instances the oligonucleotide primer or repressor protein will have, or be capable of having, a reporter molecule and so forth. The reporter molecule can be a catalyst, such as an enzyme, a polynucleotide coding for a catalyst, promoter, dye, fluorescent molecule, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, a mass tag that alters the weight of the molecule to which it is conjugated for mass spectrometry purposes, and the like.

The signal may be produced by a signal producing system, which is a system that generates a signal that relates to the presence or amount of a target polynucleotide in a medium. The signal producing system may have one or more components, at least one component being the label. The signal producing system includes all of the reagents required to produce a measurable signal. The signal producing system provides a signal detectable by external means, by use of electromagnetic radiation, desirably by visual examination. Signal-producing systems that may be employed in the present invention are those described more fully in U.S. Pat. Nos. 6,558,908, 6,251,588, 6,235,483 and 6,132,997, the relevant disclosures of which are incorporated herein by reference.

The linear arrays prepared as described above are particularly suitable for conducting hybridization reactions. Such reactions are carried out on a linear array comprising a plurality of features relating to the hybridization reactions. The linear array is exposed to liquid samples and to other reagents for carrying out the hybridization reactions. The support surface exposed to the sample is incubated under conditions suitable for hybridization reactions to occur.

After the appropriate period of time of contact between the liquid sample and the linear array, the contact is discontinued and various processing steps are performed. Following the processing step(s), the section of the linear array is moved to an examining device where the linear array is interrogated. The examining device may be a scanning device involving an optical system as discussed above.

Reading of the array may be accomplished by illuminating the array and reading the location and intensity of resulting fluorescence at each feature of the array. For example, a scanner may be used for this purpose where the scanner may be similar to, for example, the AGILENT MICROARRAY SCANNER available from Agilent Technologies Inc, Palo Alto, Calif. Other suitable apparatus and methods are described in U.S. patent applications: Ser. No. 09/846,125 “Reading Multi-Featured Arrays” by Dorsel, et al.; and U.S. Pat. No. 6,406,849. The relevant portions of these references are incorporated herein by reference. However, arrays may be read by methods or apparatus other than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,221,583 and 6,251,685, and elsewhere).

Results from the reading may be raw results (such as fluorescence intensity readings for each feature in one or more color channels) or may be processed results such as obtained by rejecting a reading for a feature that is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

When one item is indicated as being “remote” from another, this means that the two items are at least in different buildings and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

One or more embodiments of the present invention provide advantages over known apparatus and methods. In certain embodiments of the invention, reactions take place within a thin enclosed chamber wherein mixing of components is facilitated despite the small volume of the chamber. In certain embodiments of the invention, the apparatus and methods utilize a small sample volume and carry out reactions in an expedited manner. In other embodiments of the present invention, the apparatus have the capability to detect the results of chemical or biochemical reactions.

All publications and patent applications cited in this specification are herein incorporated by reference as if each individual publication or patent application were specifically and individually indicated to be incorporated by reference, except insofar as they may conflict with those of the present application (in which case the present application prevails). Methods recited herein may be carried out in any order of the recited events, which is logically possible, as well as the recited order of events.

The aforementioned description includes theories and mechanisms by which embodiments of the invention are thought to work. It should be noted, however, that such proposed theories and mechanisms are not required and the scope of the present invention should not be limited by any particular theory and/or mechanism.

Although embodiments of the foregoing invention have been described in some detail by way of illustration and example for purposes of clarity of understanding, it will be readily apparent to those of ordinary skill in the art in light of the teachings of this invention that changes and modifications may be made thereto without departing from the spirit or scope of the appended claims. Furthermore, the foregoing description, for purposes of explanation, used specific nomenclature to provide a thorough understanding of the invention. However, it will be appreciated that one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

1. An apparatus comprising:
(a) a rotatable support for one or more linear arrays, each of said linear arrays comprising an array of features for conducting chemical reactions,
(b) a rotation device for rotating said support, and
(c) an examining device for examining for the results of said chemical reactions.

2. An apparatus according to claim 1 wherein said rotatable support comprises a circular tray.

3. An apparatus according to claim 1 further comprising a heater for heating said linear arrays.

4. An apparatus according to claim 1 wherein said examining device comprises an imaging system.

5. An apparatus according to claim 1 wherein said examining device comprises a CCD detector.

6. An apparatus according to claim 1 further comprising a fluid dispensing device.

7. An apparatus according to claim 1 wherein said rotatable support comprises one or more retaining elements, each of which receives an elongated array unit in a seated position in which it extends in the radial direction while retaining the array in the seated position during rotation about an axis of the support.

8. An apparatus according to claim 1 wherein said examining device is linear and oriented so as to be optically aligned with each of multiple seated array units in turn as the support is rotated.

9. An apparatus according to claim 1 further comprising a processor that controls rotation of said support such that at one speed fluid is removed from said linear array and at another speed the support is advanced and held for examination by said examining device.

10. An apparatus for conducting and analyzing the results of hybridization reactions, said apparatus comprising:
(a) a circular tray having a surface for supporting one or more channels, each of said channels comprising one or more linear arrays, each of said linear arrays comprising a plurality of biopolymers for conducting hybridization reactions,
(b) a rotation device for rotating said circular tray, and
(c) a scanning device for examining the results of said hybridization reactions.

11. An apparatus according to claim 10 wherein said scanning device comprises an imaging system.

12. An apparatus according to claim 10 further comprising a heater for heating said linear arrays.

13. An apparatus according to claim 10 wherein said scanning device comprises a CCD detector.

14. An apparatus according to claim 10 further comprising a fluid dispensing device.

15. An apparatus according to claim 10 further comprising a bubble detecting device for examining said channels for the presence of bubbles therein.

16. A method for conducting chemical reactions, said method comprising:
(a) introducing a sample into a linear array of an apparatus according to claim 1 by capillary action,
(b) incubating said sample in said linear array under conditions for carrying out said chemical reactions, and
(c) rotating said support to remove said sample from said linear array.

17. A method according to claim 16 wherein said sample is introduced into said linear array by placing a sample at the orifice of said linear array, said linear array having internal dimensions to draw said sample therein by capillary action.

18. A method according to claim 16 wherein said linear array is rotated to remove said sample from said linear array by centrifugal force.

19. A method for conducting hybridization reactions, said method comprising:
(a) placing a sample at an orifice of a channel of an apparatus according to claim 16, said channel having internal dimensions to draw said sample therein by capillary action,
(b) incubating said sample in said channel under conditions for carrying out said hybridization reactions, and
(c) rotating said channel to remove said sample from contact with said features of said linear arrays by centrifugal force.

20. A method according to claim 19 wherein said channel is rotated in step (c) about an axis perpendicular to the axis of said channel wherein the rate of rotation about said axis is sufficient to remove said sample from contact with said features.

21. A method according to claim 19 further comprising examining said channel for the presence of bubbles therein subsequent to step (a).

22. A method according to claim 19 wherein a plurality of said channels is employed and said channels are disposed on a rotatable support.

23. A method according to claim 22 wherein said rotatable support is a circular tray.

24. A method according to claim 22 wherein said rotatable support is rotated in step (c).

25. A method according to claim 19 wherein said channel is heated prior to step (a) to a temperature for incubating said sample in step (b).

26. A method according to claim 19 further comprising introducing a wash liquid into said channel prior to or in conjunction with step (c).

27. A method according to claim 19 further comprising examining said linear array for the results of said hybridization reactions.

28. A method according to claim 19 wherein said channel is part of a microfluidic system.

29. A method according to claim 19 wherein said channel is part of a housing.

30. A method according to claim 19 wherein said linear array is a linear microarray.

31. A method according to claim 19 wherein said features are biopolymers.

32. A method according to claim 19 wherein said features are polynucleotides or polypeptides.

33. A method according to claim 19 wherein said linear array comprises at least ten features.

34. A method comprising forwarding data representing a result obtained from a method according to claim 27.

35. A method according to claim 34 wherein the data is transmitted to a remote location.

36. A method comprising receiving data representing a result obtained from a method according to claim 27.
